(19) United States
(12) Patent Application Publication
(10) Pub. No.: US 2009/0048481 A1
(43) Pub. Date: Feb. 19, 2009

(54) METHOD AND APPARATUS FOR IMPROVING CIRCULATION AND TREATING ERECTILE DYSFUNCTION

(76) Inventor: Richard S. Dillon, Bryn Mawr, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/839,758

(22) Filed: Aug. 16, 2007

(51) Int. Cl. A61F 5/00; A61B 5/02; A61H 7/00

(52) U.S. Cl. 600/41; 600/481; 601/151

(57) ABSTRACT

A method, system, and apparatus for treating erectile dysfunction by using controlled intermittent pressure cycles in synchronization with the heart beat. The apparatus comprises two boots, one encircling a patient's leg, and the other encircling the patient's penis. A pneumatic system including the pneumatic apparatus also comprises a heart monitor for recording and/or displaying the patient's heart beat and initiating the deflation and inflation of the boots at the appropriate times. The system may also include a valve assembly for controlling the flow of air into the pneumatic apparatus.

### FIELD OF THE INVENTION

The present invention relates to a pneumatic system, apparatus, and method for improving circulation of blood in a patient. The present system, apparatus, and methods are useful for treating erectile dysfunction.

### BACKGROUND OF THE INVENTION

Erectile Dysfunction (ED) is a medical condition that makes obtaining or sustaining an erection difficult or impossible. Various physical and emotional conditions cause ED, affecting over fifteen million Americans. USA TODAY, Health Encyclopedia Erectile Dysfunction. There are many different ways to treat ED including the ingestion of PDe5 which is designed to promote the inflow of blood into the penis. Additionally, a number of medical devices have been designed to treat ED. Generally, these devices utilize vacuums and constriction rings to help mitigate the symptoms of ED.

For example, U.S. Pat. No. 7,083,570 utilizes both a vacuum chamber as well as a constriction ring. U.S. Pat. No. 6,926,666 also uses a combination of a vacuum and constriction ring to operate. Most vacuum constriction system utilize negative pressure to help blood flow into the penis to promote the filling of blood into the corpus cavernous thereby creating an erection. Males suffering from ED, will often rapidly lose their erection once the vacuum chamber is removed from the penis.

To solve that problem, many of these devices use a constriction ring to prevent venous return of blood out of the penis. The blood stays pooled in the penis, and the erection is maintained for a certain time.

The constriction devices can be made from a wide variety of materials such a rubber rings or pressure chambers. U.S. Pat. No. 5,295,946 discloses a penis cuff that constricts around the penis via expandable chambers, trapping blood within the shaft of the penis.

Though these devices do help mitigate the symptoms of ED, they do not cure it. Additionally, many individuals do not want to use these devices before engaging in intercourse, because it is disruptive to the emotional connection of both partners. While the use of several medications has proved successful in treating ED, the medication is very expensive, temporary, and carries with it a host of side effects.

### SUMMARY OF THE INVENTION

An aspect of the current invention is to provide a long-lasting treatment method, system, and apparatus that does not require the male to use a device immediately before engaging in sexual activity nor require the male to take any type of medication.

The current invention includes, inter alia, an apparatus, system, and method for improving blood flow in the entire body and also in the penis. In one embodiment of the current invention, the apparatus comprises at least two pressure cuffs (boots) for applying pressure to at least one leg and to the male's penis. The boots surround the man's leg and the man's penis and provide compression forces to the surrounded regions. Air pressure is introduced into air bladders inside of the boots by way of conduits or tubes. The conduits are connected to the boots and are adapted to be attached to air pressure equipment.

When a boot and its air bladder are inflated, the boot exerts pressure or a compressive force on the surrounding tissue. During treatment, the boot remains in an inflated state for a specified period of time, which is called either a compression phase or an inflation phase. The boot and its air bladder are deflated or depressurized for a specified period of time so that the boot no longer exerts pressure on the surrounding tissue. The boot and its bladder do not apply a decompressive, negative pressure, or vacuum force against the tissue; rather the pressure emitted by the boot against the tissue is simply reduced during the relaxation or deflation phase. During the relaxation phase, all pressure on the tissue may be eliminated or the pressure may simply be reduced, depending on the embodiment.

To inflate the boots and their air bladders, the boots and air bladders must be fluidly connected to air pressure equipment. The pneumatic system comprises the pneumatic apparatus previously described and the air pressure equipment. The air pressure equipment may include an air supply, a valve assembly, a heart monitor, and a pressure monitoring assembly.

The air supply provides the air to the boots. Embodiments of the air supply include a hospital wall air supply, compressed air, or an air compressor.

The valve assembly is used to transform the constant flow of air from the air supply into a controlled intermittent air flow. More specifically, the valve assembly has two phases: an inflation phase (compression phase) and a deflation phase (relaxation phase). To initiate the inflation phase, the valve assembly is connected to the air source via a conduit. Air will flow from the air source, through the valve assembly, and through a valve assembly conduit into the large boot. To initiate a deflation phase, the valve assembly exposes the valve assembly conduit to atmospheric pressure, causing the air in the boots to escape into the surrounding atmosphere. The time periods of the two phases are controlled by the heart monitor.

The heart monitor comprises an electrocardiograph which measures the change voltage on the skin of the individual caused by the contraction of the heart. In some embodiments, the heart monitor may be electrically connected to the valve assembly. In these embodiments the heart monitor is capable of creating and sending a signal that will alter the phase of the valve assembly. The heart monitor 51 sends a signal to the valve assembly 40 causing the valve assembly 40 to switch phases. For example, to initiate the compressive phase, the monitor sends a signal to the valve assembly 40 ending the relaxation phase and beginning the inflation phase. Similarly, to initiate the relaxation phase, the monitor 51 may send a signal to the valve assembly 40 ending the inflation phase and beginning the deflation phase.

Optionally, the equipment may comprise a pressure monitoring assembly. The pressure monitoring assembly may comprise a pressure gauge for measuring boot or wall (air compressor) pressure. The assembly may also comprise tubes or passages connecting the gauges to the boots or air source to measure said pressure. The pressure monitoring assembly may be connected to one or more conduits to measure conduit pressure, or be integrated into the valve assembly or the monitor.

### BRIEF DESCRIPTION OF THE FIGURES

FIG. 1 is a diagrammatic view of a first embodiment of pneumatic system.

FIG. 2 is a diagrammatic view of the boot components of the first embodiment of the pneumatic apparatus, which is a subassembly of the pneumatic system in FIG. 1.

FIG. 3 is a diagrammatic view similar to FIG. 1 of a second embodiment of the pneumatic apparatus.

FIG. 4 is a diagrammatic view of the boot components of the second embodiment of the pneumatic system.

FIG. 5 is a diagrammatic view similar to FIG. 1 of a third embodiment of the pneumatic apparatus.

FIG. 6 is diagrammatic view of the boot components of the third embodiment of the pneumatic system.

FIG. 7 shows two graphs. The first graph illustrates the pressures applied to the patient as a function of time. The second graph shows the corresponding EKG which is used by the monitor to control the compression and relaxation phases.

FIG. 8 shows a detailed graph of the compressions phases applied to the patient as a function of time.

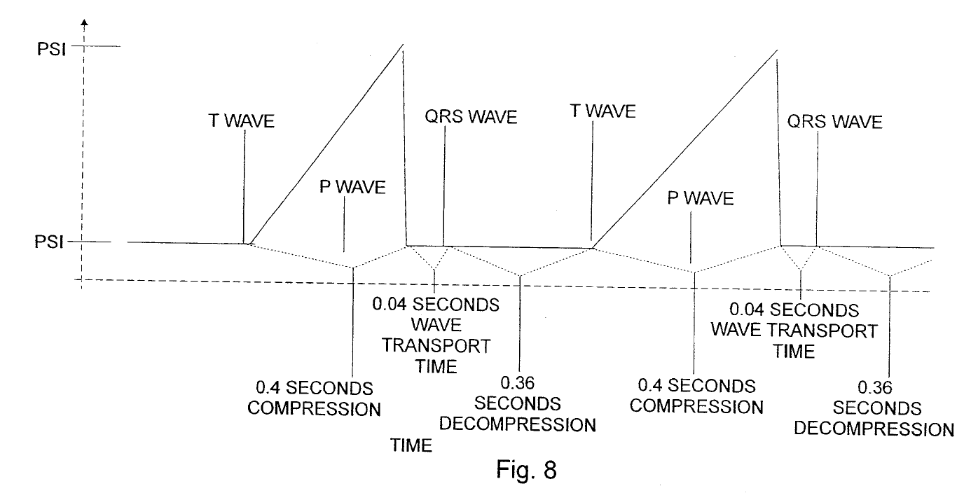

FIG. 9 shows a schematic of the valve assembly in the inflation phase as air flows through the valve assembly into the boots.

FIG. 10 shows a schematic of the valve assembly in the deflation phase as air flows from the valve assembly and escapes into the atmosphere.

FIG. 11 shows a diagrammatic view of the pressure monitoring assembly.

### DETAILED DESCRIPTION

ED can be caused by a number of conditions including diabetes, trauma, spinal nerve compression, and vascular disease. The present invention provides a method and apparatus for treating ED by increasing blood flow throughout the body. Experimentation with various systems and methods has shown that rapid compression cycles on the leg and penis of the patients creates shear forces on the endothelium in the legs and penis. These shear forces result in the elaboration of nitric oxide, VEFG (vascular endothelial growth factors), prostacyclin and fibrinolysins. Blood flow is increased through the use of a compression device which effects cyclic compressions on a patient's legs and his penis. As described in U.S. Pat. No. 5,514,079, the administration of cyclic compressions helps improve cardiac output. U.S. Pat. Nos. 3,961,625, 4,269,175, 4,343,302 and 4,590,925 (all incorporated by reference) describe suitable compression devices or boots for use in the current method, system, and apparatus.

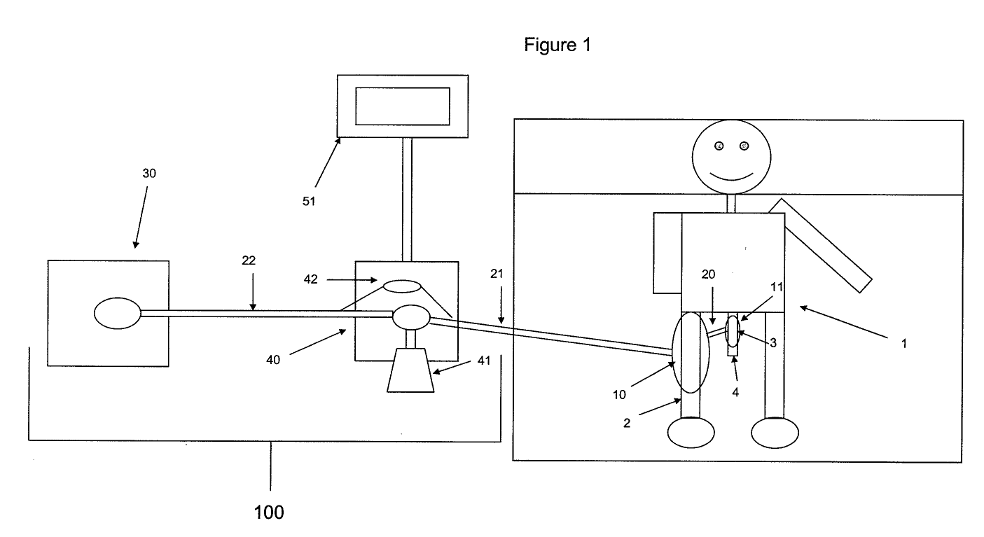

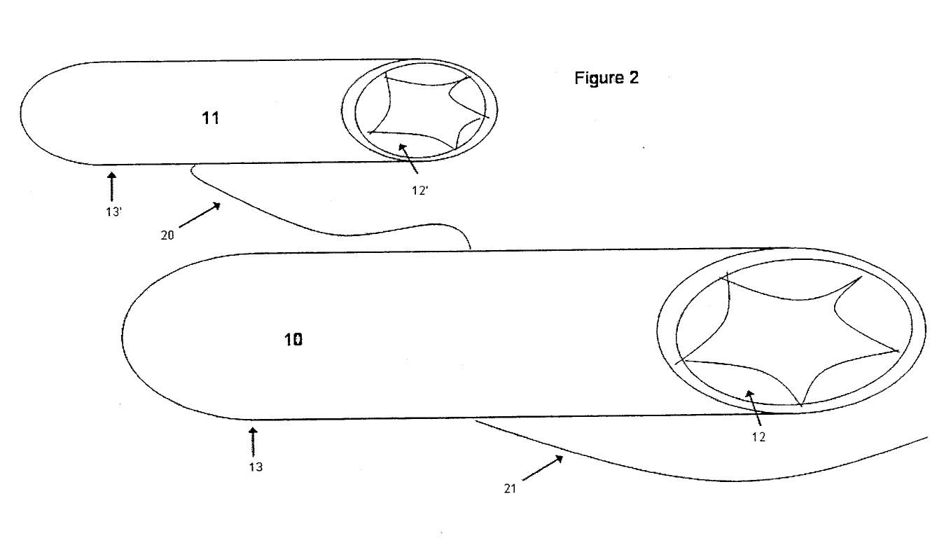

To treat a patient for ED, a health care administrator will use an embodiment of the present invention. By way of example only, three different apparatuses are shown in FIGS. 2, 3, and 5. FIGS. 1, 4, and 6 illustrate the respective pneumatic systems which are used in combination with said apparatuses. In each embodiment, a large boot 10 is applied to one leg 2 of the patient 3, and a mini boot 11 is applied to the penis 3 of the patient.

As shown in FIG. 2, in the first embodiment of the present invention, the apparatus has two conduits 20 and 21 and two boots 10 and 11. As shown in FIG. 2, each boot may comprise an outer shell 13, 13′ with an internal bladder 12, 12′. The bladder 12′ is connected to the bladder 12 by a conduit 20, and the bladder 12 is connected to the air supply through the valve assembly by a conduit 21. In other embodiments, the apparatus can comprise more than two boots, multiple bladders, or more than two conduits.

FIG. 1 illustrates a pneumatic system comprising the apparatus of FIG. 2, and air equipment 100. In this embodiment, the air equipment comprises an air pressure supply 30, a valve assembly 40, and a heart monitor 51. The air equipment introduces air into the apparatus via conduit 21. The initial source of the air pressure is generated by the air supply 30. Air flows from the air supply 30 through an air supply conduit 22 into a valve assembly 40. The air supply conduit 22 may have a first end adapted to be connected to an air supply and a second end directly connected to the valve assembly 40. In other embodiments the air supply conduit may have a first end directly connected to the air supply or a second end adapted to attach to the valve assembly 40. From the valve assembly 40, air flows through a valve assembly conduit 21 into a large (leg) boot 10. Air fills the bladder of the large (leg) boot 10, and flows out of the large (large) boot into the boot conduit 20. The air flows through the boot conduit into the second (mini) boot 11. Because the large and mini boots are fluidly connected, the large and mini boots are inflated and deflated substantially simultaneously.

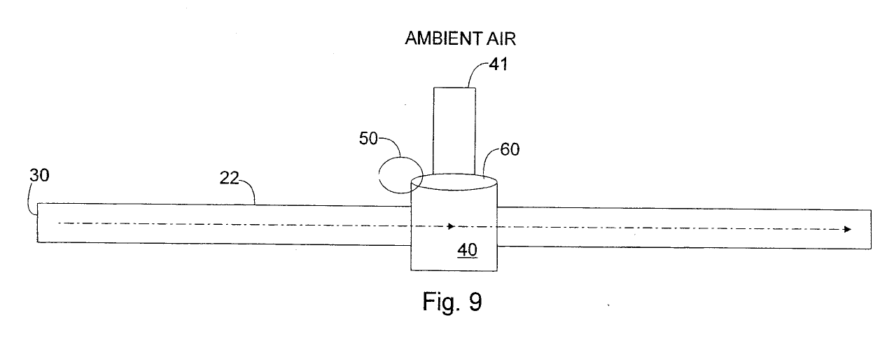

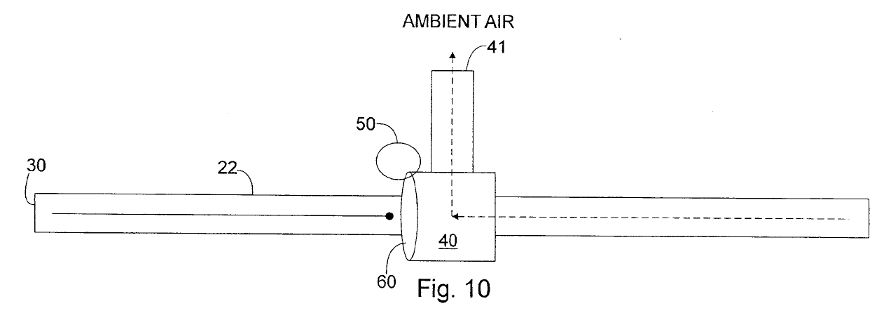

FIG. 9, illustrates the valve assembly in greater detail. To release air pressure from the pneumatic apparatus, a valve operator 50 and the heart monitor 51 causes the valve element 60 to block the flow of air from the air supply conduit 22 and connect the valve assembly conduit 21 to normal atmospheric pressure. The valve operator 50 may be controlled by heart monitor, and may cycle the valve element 60 between an open state and closed state by adjusting the position, form, or structure of the valve element. The air inside the large 10 and mini 11 boots rapidly escapes, thereby depressurizing the boots. Additionally, the valve assembly 40 may comprise an air releaser 41 (FIG. 2), which allows air to escape. The dashed arrow in FIG. 10 represents the flow of air through the valve assembly during the deflation phase. As shown schematically in FIG. 10, the valve element 60 blocks the flow of air into the valve assembly during the deflation phase. U.S. Pat. No. 4,343,302, incorporated by reference, provides greater detail and additional configuration options for constructing a valve assembly 40.

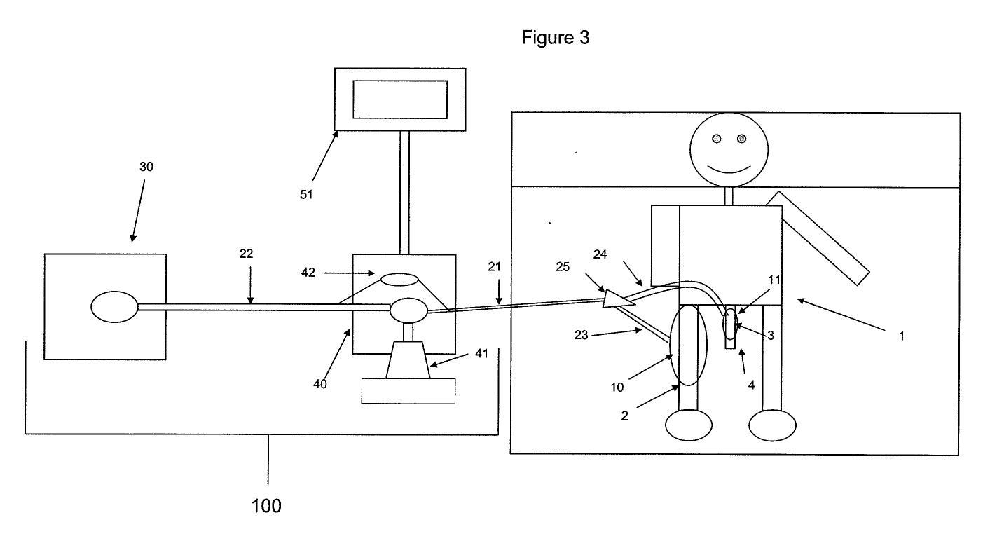

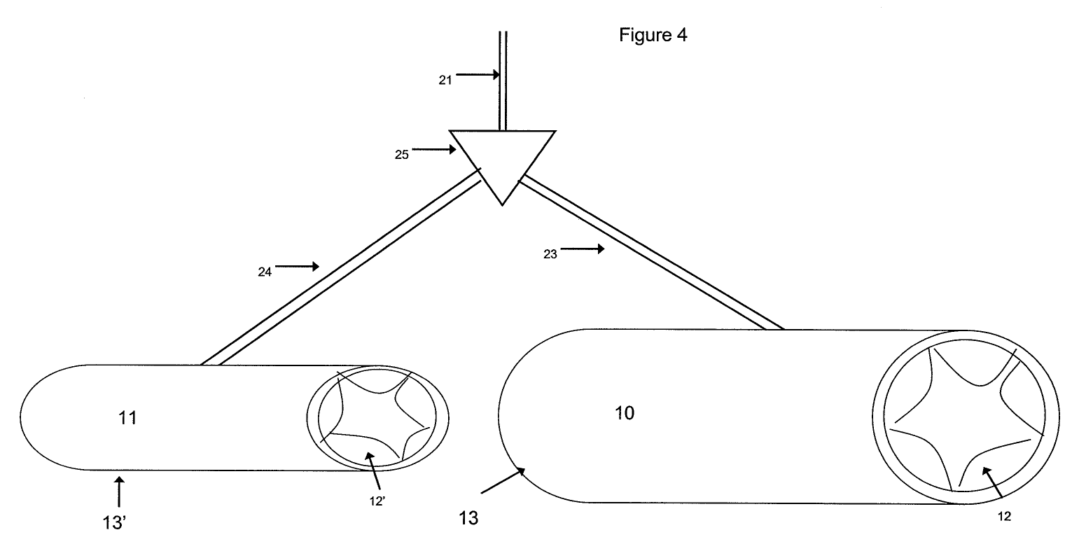

In a second embodiment of the invention, as shown in FIGS. 3 and 4, the apparatus comprises two boots 10 and 11, a valve assembly 40, four conduits 21, 22, 23, and 24, and a Y or T junction 25 which splits the air flow between the large boot conduit 23 and the mini boot conduit 24. In this embodiment, air flows from the valve assembly conduit 21 and then into the Y junction 25 and splits between the large boot conduit 23 and the mini boot conduit 24. The large boot conduit 23 supplies air to the large boot 10, and the mini boot conduit 24 supplies air to the mini boot 11 as shown in FIGS. 3 and 4. The air supply conduit 22 is designed to connect air supply 30 to valve assembly 40 to deliver air to the valve assembly conduit 21. See FIG. 4.

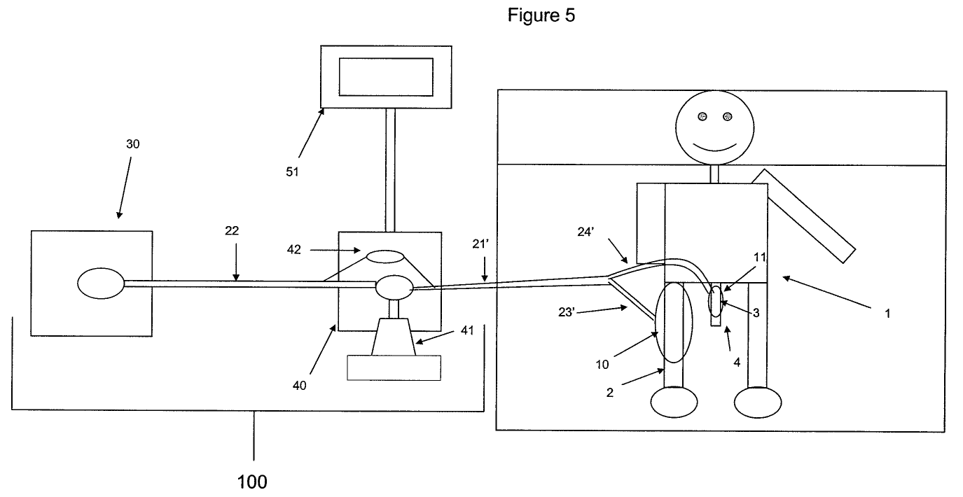

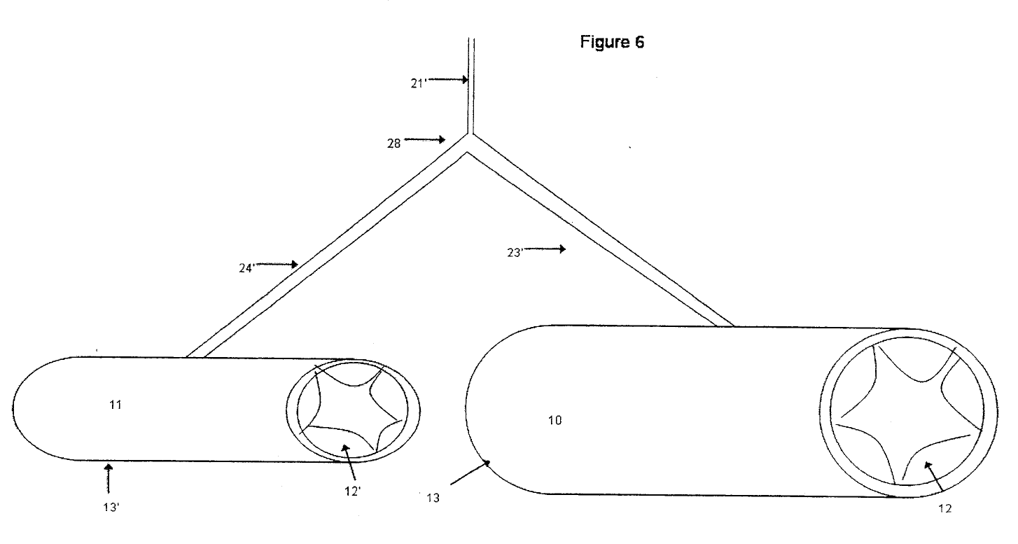

A third embodiment of the present invention is shown in FIGS. 5 and 6. This embodiment comprises a one-piece conduit 28. Said one-piece conduit comprises an input passage 21′, a first output passage 24′, and a second output passage 23′. The passages are fluidly connected so that the air flows from the input passage is split between the first and second output passages. The first output passage 23′ supplies air to the large boot 10, and the second output passage 24′ supplies air to the mini boot 11 as shown in FIG. 5. The one-piece conduit 28 is structured to connect air supply 30 to valve assembly 40 to deliver air to the input passage 21′. See FIG. 6.

To treat a patient with ED, a health care administrator or patient may use a pneumatic apparatus and system described above to perform a treatment method. A health care administrator may be a physician, nurse, or any other person familiar with the functions of the apparatus and system of the present invention.

The health care administrator or the patient 1 will apply a large boot 10 to a leg of the patient 1. Depending on the patient's condition, a boot covering the patient's entire leg and possibly his foot, may be used. A smaller boot may used if less blood circulation is desired or if an injury to the patient's leg prevents the use of a full large boot. The patient or health care administrator will apply a mini boot 11 comprising a second bladder 12′ around his penis 3. If the patient's penis 3 is flaccid, the patient 1 or health care administrator should stretch out the patient's penis 3 by gently tugging on it. With the penis 3 fully extended, the patient may apply the boot to the penis 3. The health care administrator should select boots of appropriate size for the patient 1. With respect to the mini boot 11, experimentation has shown that the boot works optimally when it extends along the length of the shaft the penis 3, but is short enough so that the glans 4 of the penis 3 remains uncovered.

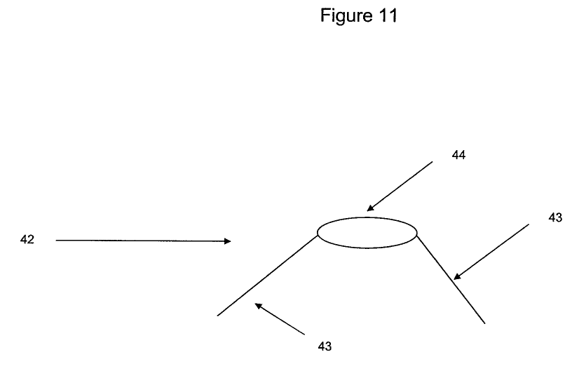

In use, the apparatus is connected to an air compressor or air supply 30, see FIG. 1. Many hospitals have air outlets which provide ready air pressure for pulmonary devices such as respirators. Alternatively, a container of pressurized air may be used. For most treatments, a pressure source capable of moving 5-15 cubic feet of air per minute should be selected. The air supply should be capable of providing a steady stream of air capable of filling the large boot 10 and mini boot 11 in about 0.4 seconds and be capable of pressuring the boots to a pressure of about 1-2 pounds per square inch. Clinical testing has shown that a boot pressure of about 1.1 PSI to be effective for treating erectile dysfunction. Boot pressure may be measured by the pressure monitoring assembly 42 shown in FIGS. 1, 4, 6, and 11. The pressure monitoring assembly 42 has at least one input 43 (FIG. 11 shows an embodiment with two inputs) for collecting air pressure data from the air supply conduit 22 or the valve assembly conduit 21. The pressure monitoring assembly 42 may also comprise a pressure gauge 44 which displays or records conduit air pressure.

The timing of the inflation and deflation phases is controlled by the heart monitor 51 which tracks the QRS complex generated by the heart. The monitor utilizes a timing cycle which is synchronized with the QRS complex to determine when to inflate and when to deflate the bladders of the boots. The heart monitor utilizes three timing elements, the time delay Td, the compression time Tc, and the negative wave transport time Wt in its timing cycle.

There are two waveforms and two wave transport times related to using compression therapy: a positive waveform having positive wave transport time and a negative waveform having a negative wave transport time. The positive waveform is created by the contraction of the heart which cause a pulse wave of blood to enter the extremities of the body. The amount of time between the contraction of the heart and the receipt of blood into the extremity is called the positive wave transport time. The negative waveform is created by the explosive decompression of the large boot which creates a well or sink in the leg allowing the blood in the aorta to fall into the leg. The amount time that occurs between the reduction of compression pressure on the leg and the drop in blood pressure to travel from the leg to the aortic valves of the heart is called the negative wave transport time. Experimentation and clinical measurements have shown the negative wave transport time to be approximately 0.04 seconds. This time period was determined by studying the effects of boot compression on the carotid and brachial waveforms. By studying the changes in the shape of the pulse waves in the brachial and carotid arties at various deflation time intervals before the QRS, it was determined that about 0.04 seconds was the most effective delay time. In some embodiments of the invention, the heart monitor 51 will continuously measure the negative wave transport time, and use the negative wave transport time as an offset before boot decompression begins, thereby allowing the negative waveform to coincide with the systolic waveform producing a narrower systolic waveform.

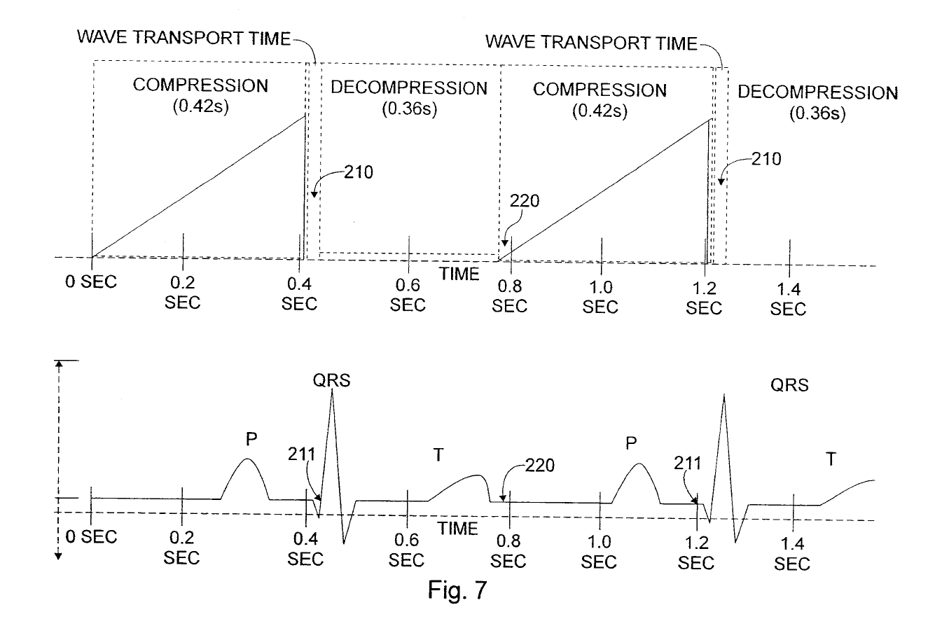

The monitor is capable of counting down the timing elements using a timer. In some embodiments, the monitor 51 may average the R-R intervals of the last 10 heart beats, and may apply a nomogram if the heart rate is irregular. The monitor may also estimate both the next R-R interval and the moment of the arrival of the next QRS complex. Based on the estimated moment of arrival of the next QRS complex, the monitor 51 may use an offset to begin deflating the boots slightly before the next QRS complex occurs. The offset would be equal the Wt. The monitor then detects the next QRS complex 211, and the monitor counts down the Td, while causing the valve assembly 40 to open and deflate the boot. After the delay time elapses (shown in FIG. 7 as element 220), the monitor 51 counts down the Tc and causes the valve assembly 40 to close, thereby inflating the boots and compressing the leg and penis. After the Tc expires (shown in FIG. 7 as element 210), the monitor 51 counts down the Wt, and causes the valve assembly to open and deflate the boot. The cycle repeats with the monitor using the offset to begin the next deflation cycle. The length of time for the Tc is about 0.38-0.44 seconds per compression. While this range can be expanded, the compression time should be long enough so that enough blood is moved, but short enough so that that blood pulse wave from the heart is not blocked by the compression of the boot. The length of time for Wt has been calculated by measuring the amount of time for the pulse wave from the heart to enter the leg which has been determined to be approximately 0.04 seconds. The Dt is calculated and is equal to the length of time for one R-R interval minus the sum of the compression time and the negative wave transport time (Td=R-R minus Tc minus Wt). Thus the average Td times range from about 0.3-0.36 seconds per contraction.

At slow heart rates, boot inflation begins after the T-wave. The QT interval is the time between the beginning of the QRS complex and the end of the T-wave. The QT interval will shorten (the T-wave occurs closer to the QRS complex) as the heart rate increases. However, at rapid heart rates (for example over 100 beats per minute where the R-R interval is about 0.6 seconds), the delay time begins to encroach on the QT interval. Thus, a Tc of 0.38 and the Wt of 0.04 add up to 0.42 leaving only 0.18 seconds for the Td. The QT interval might shorten to perhaps 0.34. In such a situation the boot would begin to squeeze the leg before systole ended, which reduces the effectiveness of the system. In practice, the heart may be slowed with a beta-blocker to increase the systems efficiency, or the compression cycles may be timed to occur on alternate beats.

Additional embodiments and alterations to the disclosed systems, apparatuses, and methods may be used. For example, U.S. Pat. No. 5,514,079, incorporated by reference, provides a description of a suitable monitor for this invention. The monitor described in U.S. Pat. No. 5,514,079 has the capability to send signals to the valve assembly causing the assembly to allow air to flow into the valve assembly conduit 21 or allow air to flow out of the valve assembly conduit 21. U.S. Pat. No. 5,514,079 also disclosed various timing cycles, which may used in conjunction with the present invention.

In other embodiments of the invention, it may not be desirable to apply and remove pressure on every heart beat. Depending on the needs of the patient or his treatment, an inflation cycle can be performed every other R-R cycle or once every three cycles. Also, the number of treatment cycles per day can be varied as necessary for the patient. For most conditions, about an hour long treatment will be desirable. The number of boot compressions per hour will vary depending a patient's pulse rate. For example if a person's pulse rate is 50 beats per minute, the boot will operate at the rate of 50 compressions per minute or 3000 compressions per hour. Additionally, for those patients requiring one compression every second, third, or fourth heart beat, the number of compressions per minute would be reduced by a half, third, or fourth respectively. For example, for a patient with a pulse rate of 100 and modest peripheral artery disease, a 2:1 compression setting may be used leading to 50 compressions per minute, for a total rate of 3000 compression per hour. For average pulse rates, 60-120, the average compression times would range from 3,600 compressions per hour (60 compressions per minute times 60 minutes/hour) to 7,200 (60 compressions per minute times 120 minutes/hour). If a 2:1 compression setting is being used, the number of compressions would range from 1,800-3,600 compressions per hour.

Additional embodiments of the apparatus and methods of using the present invention are contemplated. Additional embodiments may be built and used with different compression devices or different valve assemblies. In accordance with invention, the large boot is applied to a portion of the body with a large surface area, and the mini boot is applied to an extremity which has a smaller surface area. The current invention may be used to treat conditions other than erectile dysfunction, which may be related to poor blood circulation. For example, an embodiment of the present invention may be used to treat tennis elbow. The foregoing embodiments are exemplary only and no limitations to the current invention are intended except as provided in the following claims. It is claimed.

1. A pneumatic apparatus adapted to be connected to air equipment and useful for treating erectile dysfunction in a patient, said apparatus comprising:
a. A large boot comprising a first, inflatable, air bladder; said boot structured to surround the leg of said patient to allow controlled intermittent pressure to be applied to the leg;
b. A mini boot comprising a second, inflatable, air bladder; said mini boot structured to surround the penis of said patient to allow intermittent pressure to be applied to the penis;
c. A valve assembly conduit attached to the large boot and designed to be fluidly connected to the air equipment for allowing air to pass through the conduit and inflate said first air bladder;
d. A mini boot conduit attached to the large and mini boots; said mini boot conduit allowing air to flow from the large boot into the mini boot.

2. A pneumatic system for treating erectile dysfunction in a patient, said system comprising:
a. The pneumatic apparatus according to claim 1;
b. A valve assembly fluidly connected to the large boot by the valve assembly conduit, capable of controlling the flow of air into and out of the boots, and comprising a valve element for periodically blocking the flow of air through the valve assembly, and a valve operator that cycles the valve element between an open state and closed state;
c. A heart monitor electrically connected to the valve assembly; said monitor capable of issuing a signal that will alter the phase of the valve assembly by causing the valve operator to adjust the positioning of the valve element; and
d. An air supply conduit for delivering air from an air supply to the valve assembly; said air supply conduit comprising:
i. A first end designed to be fluidly connected to an air supply; and
ii. A second end fluidly connected to the valve assembly for delivering air from the air supply to the valve assembly.

3. The apparatus of claim 2 comprising a pressure monitoring assembly fluidly connected to the valve assembly conduit, said monitoring assembly comprising an air pressure gauge for determining the air pressure in the large boot.

4. A pneumatic apparatus adapted to be connected to air equipment and useful for treating erectile dysfunction in a patient, said apparatus comprising:
a. A large boot comprising a first, inflatable, air bladder; said boot structured to surround a leg of the patient to allow intermittent pressure to be applied around the leg;
b. A mini boot comprising a second, inflatable, air bladder; said mini boot structured to surround the penis of said patient to allow intermittent pressure to be applied around the penis; and
c. A conduit with an input passage, a first output passage, and a second output passage; said input passage structured to be fluidly connected to the air equipment; said first output passage fluidly connected to said large boot for allowing air from the air supply to pass through the conduit and inflate the large boot; said second output passage fluidly connected to the mini boot for allowing air from the air supply to pass through the conduit and inflate the mini boot.

5. A pneumatic system for treating erectile dysfunction in a patient, said system comprising:
a. The pneumatic apparatus according to claim 4;
b. A valve assembly fluidly connected to the large boot by the valve assembly conduit, capable of controlling the flow of air into and out of the boots, and comprising a valve element for periodically blocking the flow of air through the valve assembly, and a valve operator that cycles the valve element between an open state and closed state;
c. A heart monitor electrically connected to the valve assembly; said monitor capable of issuing a signal that will alter the phase of the valve assembly by causing the valve operator to adjust the positioning of the valve element; and
d. An air supply conduit for delivering air from an air supply to the valve assembly; said air supply conduit comprising:
i. A first end designed to be fluidly connected to an air supply; and
ii. A second end fluidly connected to the valve assembly for delivering air from the air supply to the valve assembly.

6. The apparatus of claim 5 comprising a pressure monitoring assembly fluidly connected to said conduit, said monitoring assembly comprising an air pressure gauge for determining the air pressure in the large boot.

7. A method of treating erectile dysfunction in a patient by increasing blood circulation throughout the body and providing controlled intermittent compressive forces on both the leg and penis of the patient, said method comprising the steps of:
a. Mounting a large boot having an inflatable bladder to surround a patient's leg;
b. Mounting a mini boot having an inflatable bladder to surround the patient's penis; and
c. Administering controlled intermittent air pressure to inflate the bladders of the large and mini boots to apply pressure concurrently to the leg and penis of the patient; said intermittent air pressure alternately:
i. Causing the inflation of the bladders of the boots after a first QRS complex of the heart is detected, thereby providing compression forces on the leg and penis of the patient for a first period of time, and
ii. Causing the deflation of the bladders before the next QRS complex of the heart is detected, thereby reducing the compression forces on the leg and penis of the patient for a second period of time.

8. The method of claim 7, wherein the first period of time is about 0.4 seconds; and the second period of time is about 0.36 seconds.

9. The method of claim 7, comprising the steps of inflating and deflating the bladders once every heartbeat.

10. The method of claim 7, comprising the steps of inflating and deflating the bladders at a rate of between 1,800-3,600 times per hour.

11. The method of claim 7, comprising the steps of inflating and deflating the bladders at a rate of between 3,600-7,200 times per hour.

12. The method of claim 7, wherein the step of mounting a large boot comprises mounting said large boot around the entire leg of the patient.

13. The method of claim 7, wherein the step of mounting said mini boot comprises mounting said mini boot around the shaft of the penis while leaving the glans of the penis uncovered.

14. The method of claim 7, comprising the step of inflating the large boot to a pressure of about 1.1 pounds per square inch.

15. The method of claim 7, comprising the steps of:
a. Monitoring voltage waves generated by the patient's heart; said voltage waves including the QRS complex;
b. Utilizing a compression time and delay time;
c. Causing a heart monitor to deflate the bladders of the boots and begin counting down the delay time once the monitor detects the QRS complex;
d. Inflating the bladders of the boots once the delay time elapses;
e. Causing the heart monitor to maintain the bladders in an inflated for a period of time equal to the compression time; and
f. Deflating the bladders once the compression time elapses.

16. The method of claim 15, comprising the step of repeating steps b-f at a rate of about three-thousand times an hour.

17. The method of claim 15, comprising the steps of:
a. Utilizing a negative wave transport time; and
b. Causing the monitor to use an offset to begin counting down the delay time before the QRS complex occurs; the offset being equal to the negative wave transport time.
